(19) United States
(12) Patent Application Publication
(10) Pub. No.: US 2007/0149911 A1
(43) Pub. Date: Jun. 28, 2007

(54) Woven brace

(76) Inventor: Diana K. Linneman, Town and Country, MO (US)

Correspondence Address: GREENSFELDER HEMKER & GALE PC, SUITE 2000, 10 SOUTH BROADWAY, ST LOUIS, MO, 63102, US

(21) Appl. No.: 11/315,083

(22) Filed: Dec. 22, 2005

(51) Int. Cl. A61F 13/06

(52) U.S. Cl. 602/61

(57) ABSTRACT

A woven brace for use in supporting various portions of a body is disclosed. The woven brace comprises a woven body comprising a plurality of ring members woven together such that the woven brace provides a resilient and comfortable bracing surface.

### FIELD

The present document relates to a woven brace and method of assembly thereof.

### SUMMARY

In an embodiment, the woven brace comprises a woven body, the woven body including a plurality of ring members assembled together, each of the ring members having a ring body with opposing end portions and opposing middle portions, the opposing middle portions of each of the plurality of ring members being adapted to be folded such that a pair of arms with slots are defined with the ring body of one ring member being adapted to be engaged through the slots of another ring member when assembled together, wherein two of the plurality of ring members being configured as an end ring member having a closed ring configuration, wherein the middle ring members are interposed between the end ring members.

In another embodiment, a method of assembling a woven body comprises:

a) providing three or more ring members, with two of the three or more ring members being an end ring member having a closed ring configuration and the other of the three or more ring members being middle ring members, the three or more ring members having opposing end portions and opposing side portions;

b) elongating and folding the end ring member at the opposing middle portions such that first and second arms are formed with a slot defined between the first and second arms;

c) elongating and folding a first middle ring member at the opposing middle portions such that first and second arms are formed with a slot defined between the first and second arms;

d) inserting the first and second arms of the end ring member through the slot of the middle ring member such that the opposing end portions contact;

e) securing the opposing end portions of the end ring member together such that the end ring member is engaged to the first middle ring member;

f) elongating and folding a second end ring member such that first and second arms are formed with a slot defined between the first and second arms;

g) inserting the first and second arms of the second end ring member through the slot of the first middle ring member; and

h) securing the opposing end portions of end ring members to a last of the middle ring members.

Additional features will be set forth in the description which follows or will become apparent to those skilled in the art upon examination of the drawings and detailed description which follows.

### BRIEF DESCRIPTION OF THE DRAWINGS

FIG. 1 is a perspective view of a ring member for making a woven brace;

FIG. 2A is a perspective view of an outer ring material for making the ring member;

FIG. 2B is a perspective view of an inner ring material for making the ring member;

FIG. 3 is a flow chart illustrating the steps of manufacturing the ring member;

FIGS. 3A-3F are perspective views illustrating the method for manufacturing a ring member shown in FIG. 3;

FIG. 4 is a flow chart illustrating the steps of assembling the ring members to produce a woven brace;

FIGS. 4A, 4B and 4C illustrate a sequence of assembling the woven brace shown in FIG. 4;

FIG. 5 is a perspective view of the woven brace; and

FIG. 6 is a perspective view of the woven brace shown in a closed position.

Corresponding reference characters indicate corresponding elements among the view of the drawings.

### DETAILED DESCRIPTION

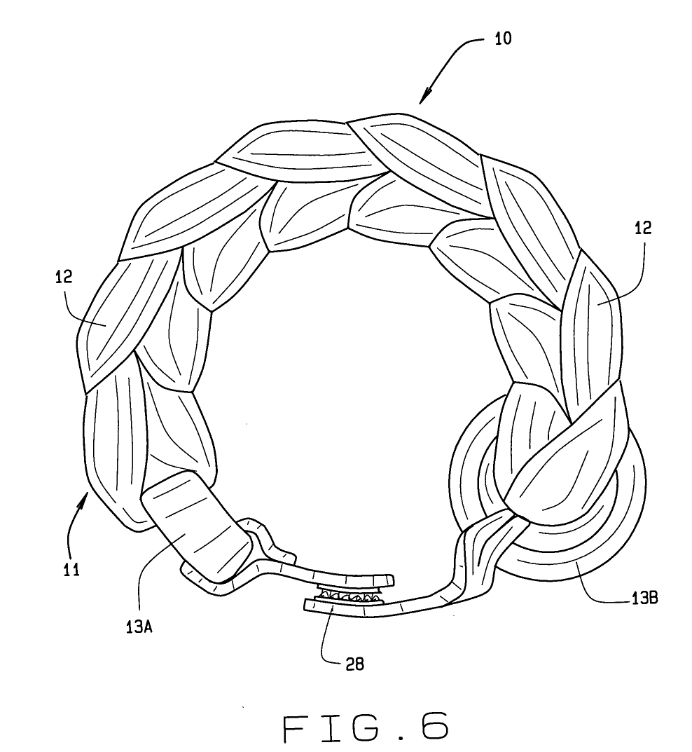

Referring to the drawings, an embodiment of the woven brace is illustrated and generally indicated as 10 in FIGS. 1-6. Referring to FIG. 5, the woven brace 10 includes a woven body 11 having a plurality of middle ring members 12 that are woven or assembled together and interposed between end ring members 13. In one embodiment, the middle ring members 12 and end ring members 13 may be identical in structure with the exception that end ring members 13 may have a closed ring configuration as shall be discussed in greater detail below. The woven body 11 may be manufactured using a method of assembly that weaves together a plurality of middle ring members 12 between two end ring members 13 to produce woven brace 11.

In one embodiment, woven brace 10 may be adapted for contacting certain portions of the user's body, such as the neck, lower back, and thighs. In addition, the woven brace 10 may be woven to provide a resilient bracing surface for supporting certain portions of the user's body.

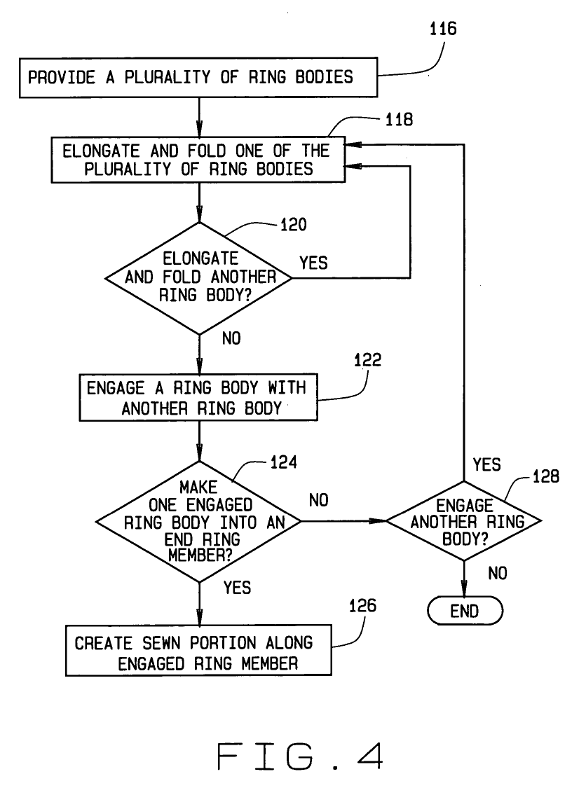

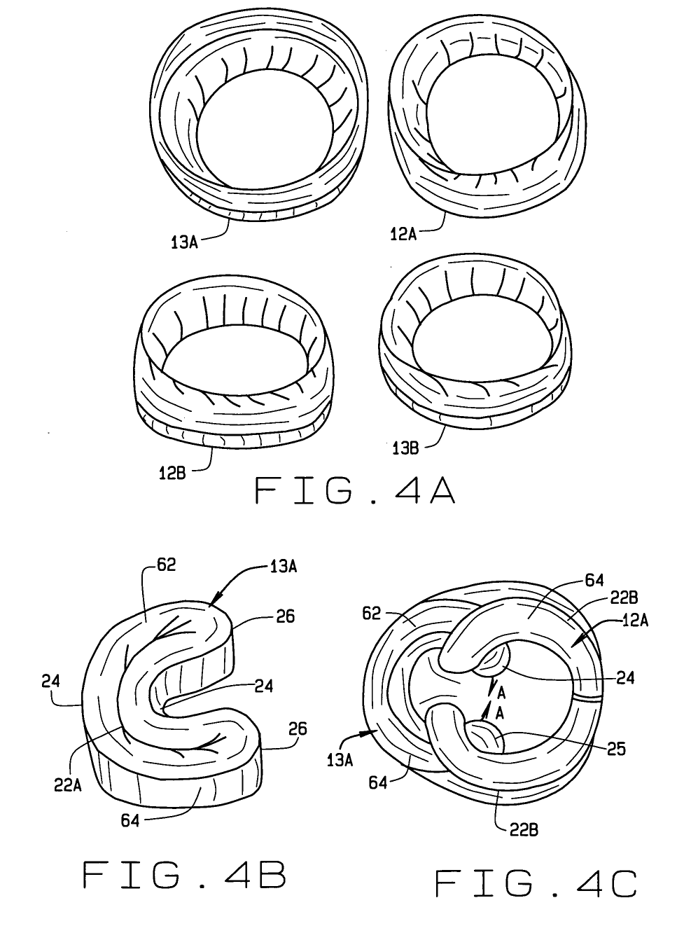

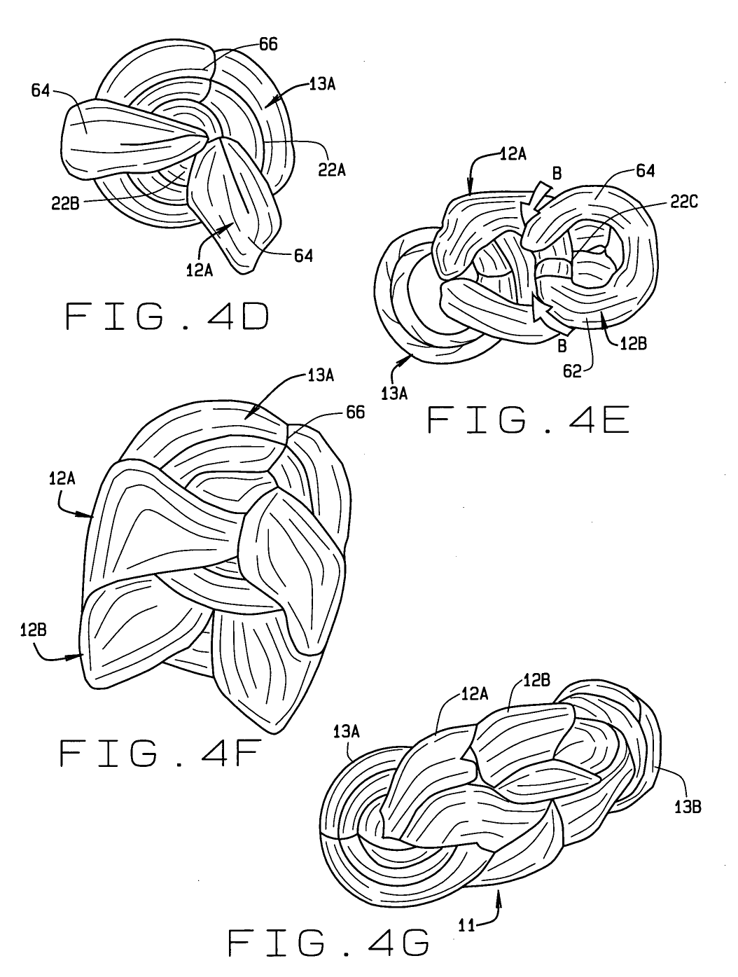

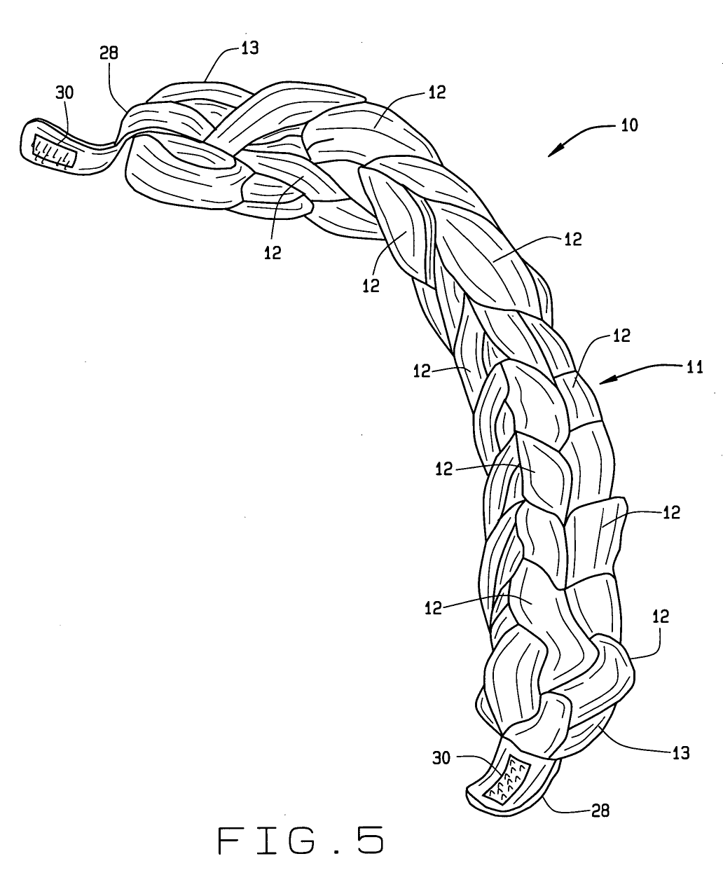

Referring to FIG. 1, middle ring members 12 and end ring members 13 may each include a ring body 14 having opposing middle portions 24 and opposing end portions 26 that collectively define a ring opening 20. Ring body 14 may further include a seam line 18 that runs along the peripheral length of ring body 14 and is produced during the manufacture of the ring member 12 as shall be discussed in greater detail below. As noted above, end ring members 13 may have the same configuration as the middle ring members 12 with the exception that the ring body 14 of the end ring members 13 may have the opposing end portions 26 sewn together to form a sewn portion 66 (FIG. 4D) during the method of assembly.

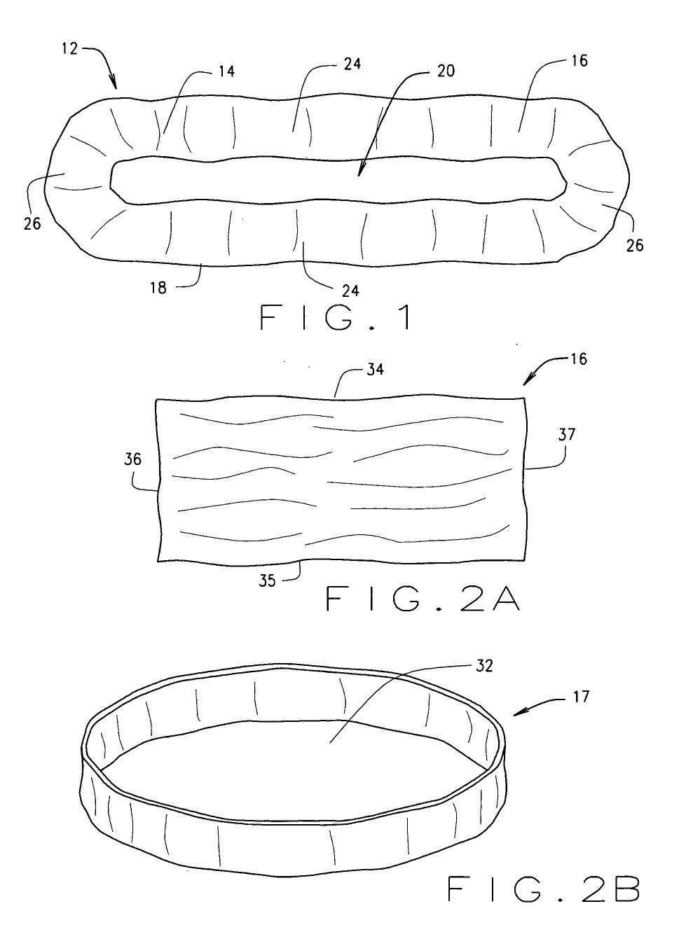

Referring to FIGS. 2A and 2B, each ring body 14 may include an outer ring material 16 defined by opposing top and bottom edges 34 and 35, and opposing side edges 36 and 37. Ring body 14 may be made for either middle ring members 12 or end ring members 13. Outer ring material 16 may be made from a fleece material, although any stretchable and soft material is contemplated, for example, T-shirt knits, poly knits, cotton, satin or fur.

In addition, ring body 14 may include an inner ring material 17 disposed inside the outer ring material 16 during manufacture. In one embodiment, the inner ring material 17 may be a webbed nylon material having a closed ring configuration defining an opening 32. However, other materials other than nylon, such as polyester fiber fill or cotton are contemplated. In addition, rice may be used as an inner ring material 17.

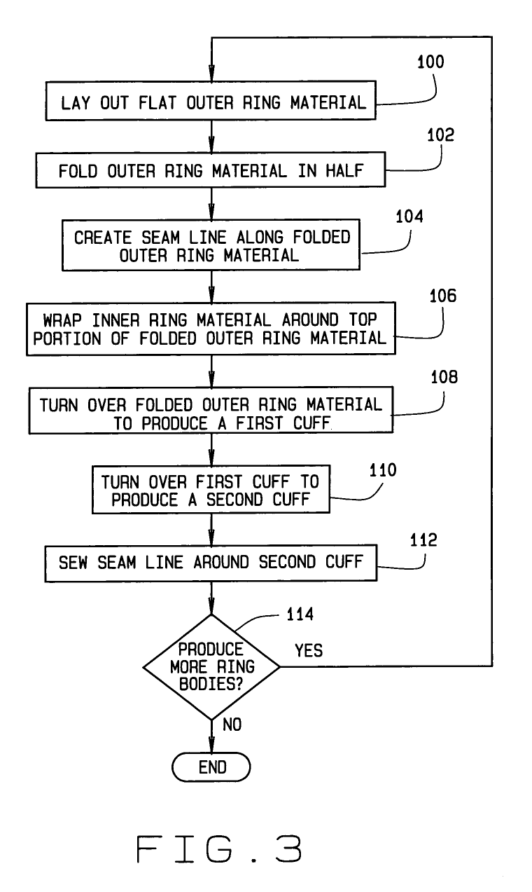

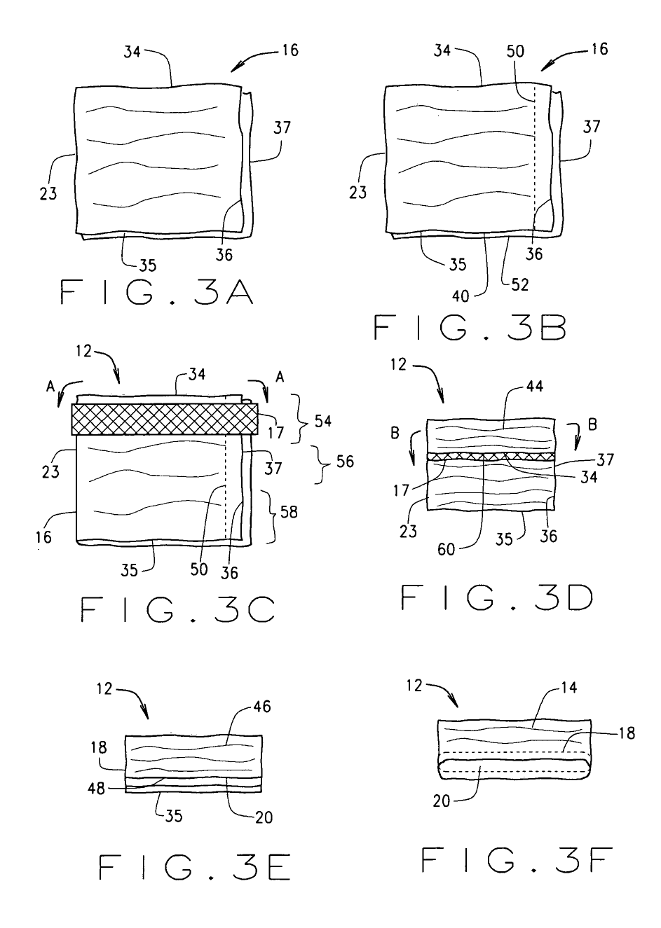

In one aspect, a method of assembling the woven brace 10 begins with manufacturing one or more ring bodies 14 in order to produce middle ring members 12 and end ring members 13. As illustrated in the flowchart of FIG. 3, each middle ring member 12 and end ring member 13 may be manufactured by laying out flat the outer ring material 16 (FIG. 2A) at step 100.

At step 102, the outer ring material 16 may be folded in half such that the opposing side edge 36 meets the opposing side edge 37 and a folded edge 23 is defined as illustrated in FIG. 3A. In this configuration, the outer ring material 16 is defined by an upper portion 54, middle portion 56 and lower portion 58.

At step 104, a seam line 50 may then be made by sewing the opposing side edge 36 to the opposing side edge 37 such that an open pocket 40 in communication with an opening 52 is defined as shown in FIG. 3B. Once the outer ring material 16 is folded in half and the seam line 50 sewn, inner ring material 17 may be wrapped around the upper portion 54 of the folded outer ring material 16 at step 106 such that the opposing top edge 34 may be inserted through opening 32 of the inner ring material 17 as illustrated in FIG. 3C.

At step 108, the upper portion 54 of the folded outer ring material 16 may be turned over upon the middle portion 56 such that a first cuff 44 is defined (FIG. 3D) with the inner ring material 17 enveloped within first cuff 44 and in communication with a cuff opening 60.

First cuff 44 may then be turned over upon the lower portion 58 at step 110 such that a second cuff 46 is defined as shown in FIG. 3E so that an open end 48 and ring opening 20 are defined by the outer ring material 16. Once the second cuff 46 is defined, a seam line 18 may be sewn at step 112 along the peripheral portion of open end 48 such that ring body 14 is defined as illustrated in FIG. 3F.

After the ring body 14 is manufactured in accordance with steps 100-112, a decision point 114 may be reached to determine whether more ring bodies 14 are required for producing either more middle ring members 12 and/or end ring members 13. If more ring bodies 14 need to be produced, then steps 100-112 may be repeated as desired; however, if no further ring bodies 14 need to be produced then the method of manufacturing ring bodies 14 is complete.

In one embodiment, the method of assembling the woven body 11 may utilize three or more ring bodies 14 with two of the ring bodies 14 being configured as end ring members 13 and the remainder being middle ring members 12 as shall be discussed below. However, it is contemplated that any number of middle ring members 12 may be manufactured and added between the two end ring members 13 during the assembly method for making different lengths of woven brace 10.

Referring to FIG. 4, a flow chart illustrating one embodiment of the method of assembling the woven body 11 is shown. For the purpose of illustrating the method of assembly only two end ring members 13A and 13B and two middle ring members, designated 12A and 12B, will be discussed as shown in FIG. 4A. However, as illustrated in FIG. 5, the woven brace 10 may have any desired number of middle ring members 12 woven together between the end ring members 13 in order to assemble a woven body 11 of sufficient length.

At step 116, woven body 11 made in accordance with the method of assembly by providing a sufficient number of ring bodies 14 to assemble a woven brace 10 of predetermined length. At step 118, one of the ring bodies 14, such as ring body 14 of end ring member 13A, may be elongated and then folded at the opposing middle portions 24 such that arms 62, 64 having a slot 22A defined along each arm 62, 64 as illustrated in FIG. 4B.

Once end ring member 13A is folded, a decision point 122 is reached to determine whether another ring body 14 should be elongated and folded. If another ring body 14 needs to be added, the method of assembly returns to step 118 where middle ring member 12A may then be similarly elongated and folded along the opposing middle portions 24 such that a slot 22B is defined along arms 62B, 64B of ring member 12A.

If no further ring bodies 14 are to be elongated and folded at this point in the method of assembly, then the method proceeds to step 122 where the arms 62, 64 of ring member 13A are then inserted through slot 22B of ring member 12A in direction A as shown in FIG. 4C. Once ring member 13A is so engaged to ring member 12B, at decision point 124, a decision must be made on whether to make ring member 13A into an end ring member 13. If so, the opposing end portions 24 of ring member 13A may be sewn together at step 126 to form a sewn portion 66 such that ring member 13A is now configured as an end ring member 13 having a closed ring configuration as illustrated in FIG. 4D. In this manner, the middle ring member 12A may be engaged to the end ring member 13A to form the first portion of the woven body 11.

Once step 126 is completed, the method proceeds to decision point 128 to determine whether another ring body 14 should be added to the woven body 11 being assembled. If another ring body 14 is to be assembled, the method of assembly proceeds back to step 118 in order to elongate and fold middle ring member 12B. Once the ring body 14 of middle ring member 12B is elongated and folded along the opposing middle portions 24 such that a slot 22C is formed along arms 62, 64 of ring member 12B. Arms 62, 64 of middle ring member 12B may then be inserted through the slot 22B of ring member 12A in direction B in a manner that orients folded ring member 12B at approximately a 90 degree angle relative to the orientation of ring member 12A as illustrated in FIG. 4E. In this manner, each additional middle ring member 12 that may be woven to the existing woven middle ring member 12A, 12B will be oriented at alternating 90 degree angles.

Once middle ring member 12B is engaged to middle ring member 12A, as shown in FIG. 4F, decision point 124 is again reached to determine whether a sufficient number of middle ring members 12 have been assembled if the woven body 11 has reached sufficient length. Therefore, the method of assembly proceeds directly to decision point 128 to determine whether another ring body 14 needs to be added.

As noted above, this particular embodiment has only two end ring members 13A, 13B and two middle ring members 12A, 12B for purposes of illustrating the basic sequence of assembling the woven body 11. As such, a larger number of middle ring members 12 to be added would require repeating the above steps until the other end ring member 13B is engaged to the last assembled middle ring member 12.

If the last middle ring member 12 has been added, then the method of assembly returns to step 118 where the ring body 14 of end ring member 13B may be elongated and folded along opposing middle portions 24 such that a slot 22D is defined along arms 62, 64 of end ring member 13B. Once so folded, the method of assembly proceeds through decision point 120 to step 122 where the arms 62, 64 of end ring member 13B may then be inserted through the slot 22C of middle ring member 12B in direction B as illustrated in FIG. 4G. At decision point 124, the opposing end portions 26 may then be sewn together such that end ring member 13B has a closed ring configuration identical to end ring member 13A. Once end ring member 13A is sewn together, the method of assembly proceeds to decision point 128 where no further ring bodies 14 need be provided. As such, the method of assembling the woven body is complete.

As noted above, the procedure of adding more middle ring members 12 may be repeated as many times as desired to assemble a woven body 11 of a desired length. In one embodiment, the length of the woven body 11 may be approximately twenty one inches. If the user desires to finish the woven body 11, the arms 62, 64 of end ring member 13B may be sewn together to form sewn portion 66. End ring member 13B now has the same closed ring configuration of end ring member 13A with middle ring members 12A and 12B being woven between end ring members 13A and 13B.

As noted above, the above description of the method of assembling the woven body 11 for only two middle ring members 12 and two end ring members 13 is for illustration purposes only and that more middle ring members 12 may be added to the existing middle ring members 12 to make a longer woven body 11.

In one embodiment, once a specific length of the woven body 11 has been assembled together, an attachment member 28 may be engaged to each end ring member 13 as illustrated in FIG. 5. This is accomplished by wrapping the body of the attachment member 28 around the end ring member 13 and sewing the free ends of the attachment member 28 together. As further shown, each attachment member 28 may include a flap 68 having a VELCRO portion 30 for engaging the VELCRO portion 30 of the other attachment member 28 in order to place the woven brace 10 in a closed configuration.

It is contemplated that the woven brace 10 may be used in different ways to brace different portions of the user's body. For example, the woven brace 10 may be placed behind the neck in order to brace the neck, or behind the lower back, or either around or below the thighs of the user. However, the woven brace 10 may be adapted to brace or cushion any portion of the user's body. In one embodiment, a woven brace 10 without attachment members 28 is contemplated.

The method of manufacturing the ring bodies 14 as well as the method of assembling the ring bodies 14 to make a woven body 11 may be accomplished either manually by hand or using an automated system that utilize one or more apparatuses to automatically manufacture and assemble the ring bodies 14 to produce the woven body 11 as described in FIGS. 3 and 4.

It should be understood from the foregoing that, while particular embodiments have been illustrated and described, various modifications can be made thereto without departing from the spirit and scope of the invention as will be apparent to those skilled in the art. Such changes and modifications are within the scope and teaching of this invention as defined in the claims appended hereto.

What is claimed:

1. A woven brace comprising:
a woven body, said woven body including a plurality of ring members assembled together, each of said ring members having a ring body with opposing end portions and opposing middle portions, said opposing middle portions of each of said plurality of ring members being adapted to be folded such that a pair of arms with slots are defined with the ring body of one ring member being adapted to be engaged through the slots of another ring member when assembled together, wherein two of said plurality of ring members being configured as an end ring member having a closed ring configuration, wherein the middle ring members are interposed between the end ring members.

2. The woven brace according to claim 1, wherein the closed ring configuration of said end ring member is made by engaging the opposing end portions together.

3. The woven brace according to claim 2, wherein the opposing end portions are engaged together by sewing the opposing end portions together.

4. The woven brace according to claim 3, wherein the sewn opposing end portions form a sewn portion.

5. The woven brace according to claim 1, wherein said ring body includes an outer ring material that envelopes an inner ring material.

6. The woven brace according to claim 5, wherein the outer ring material is made from a soft and resilient material.

7. The woven brace according to claim 6, wherein the soft and resilient material is a fleece material.

8. The woven brace according to claim 5, wherein the inner ring material is made from a nylon material.

9. The woven brace according to claim 1, wherein each of the end ring members are adapted to engage a respective attachment member, each respective attachment member being adapted to engage the other respective attachment member in order to place the woven brace in a closed configuration.

10. The woven brace according to claim 9, wherein said attachment member comprises a flap including a VELCRO portion.

11. A method of assembling a woven body comprising:
a) providing three or more ring members, with two of the three or more ring members being an end ring member having a closed ring configuration and the other of the three or more ring members being middle ring members, the three or more ring members having opposing end portions and opposing side portions;
b) elongating and folding the end ring member at the opposing middle portions such that first and second arms are formed with a slot defined between the first and second arms;
c) elongating and folding a first middle ring member at the opposing middle portions such that first and second arms are formed with a slot defined between the first and second arms;
d) inserting the first and second arms of the end ring member through the slot of the middle ring member such that the opposing end portions contact;
e) securing the opposing end portions of the end ring member together such that the end ring member is engaged to the first middle ring member;
f) elongating and folding a second end ring member such that first and second arms are formed with a slot defined between the first and second arms;
g) inserting the first and second arms of the second end ring member through the slot of the first middle ring member; and
h) securing the opposing end portions of end ring members to a last of the middle ring members.

12. The method of assembling according to claim 11, further comprising repeating steps f) and g) to produce a woven body of predetermined length.

13. A method of using a woven brace comprising:
a) providing the woven brace, said woven brace including a woven body having a plurality of ring members assembled together, each of said ring members having a ring body with opposing end portions and opposing middle portions, said opposing middle portions of each of said plurality of ring members being adapted to be folded such that a pair of arms with slots are defined with the ring body of one ring member being adapted to be engaged through the slots of another ring member when assembled together, wherein two of said plurality of ring members being configured as an end ring member having a closed ring configuration, wherein the middle ring members are engaged together and interposed between the end ring members; and
b) placing the woven brace around a neck of a person.

14. A method of using a woven brace comprising:
a) providing the woven brace, said woven brace including a woven body having a plurality of ring members assembled together, each of said ring members having a ring body with opposing end portions and opposing middle portions, said opposing middle portions of each of said plurality of ring members being adapted to be folded such that a pair of arms with slots are defined with the ring body of one ring member being adapted to be engaged through the slots of another ring member when assembled together, wherein two of said plurality of ring members being configured as an end ring member having a closed ring configuration, wherein the middle ring members are engaged together and interposed between the end ring members; and
b) placing the woven brace around a thigh of a person.

15. A method of using a woven body comprising:
a) providing the woven brace, said woven brace including a woven body having a plurality of ring members assembled together, each of said ring members having a ring body with opposing end portions and opposing middle portions, said opposing middle portions of each of said plurality of ring members being adapted to be folded such that a pair of arms with slots are defined with the ring body of one ring member being adapted to be engaged through the slots of another ring member when assembled together, wherein two of said plurality of ring members being configured as an end ring member having a closed ring configuration, wherein the middle ring members are engaged together and interposed between the end ring members; and
b) placing the woven brace behind the lower back of a person.
